(19) United States
(12) Patent Application Publication
(10) Pub. No.: US 2016/0030343 A1
(43) Pub. Date: Feb. 4, 2016

(54) PREPARATION AND CHARACTERIZATION OF BONE-TARGETED VANCOMYCIN-LOADED LIPOSOMES FOR OSTEOMYELITIS TREATMENT

(71) Applicant: Texas Tech University System, Lubbock, TX (US)

(72) Inventors: Amr Abdelgawad, El Paso, TX (US); Eman Atef, Cupertino, CA (US); Basel Karzoun, Boston, MA (US)

(73) Assignee: Texas Tech University System

(21) Appl. No.: 14/815,360

(22) Filed: Jul. 31, 2015

Related U.S. Application Data
(60) Provisional application No. 62/031,989, filed on Aug. 1, 2014.

(51) Int. Cl. A61K 9/127; A61K 38/14

(52) U.S. Cl. CPC A61K 9/1271; A61K 38/14

(57) ABSTRACT

A treatment method, system, and compound comprise directly targets osteomyelitis-infected bone tissue with one or more surface-modified liposomes. The surface-modified liposome(s) includes an alendronate targeting moiety utilized to modify the surface of the liposome. Alendronate targets hydroxyapatite in bone tissue, wherein the surface-modified liposome further comprises encapsulated vancomycin that directly targets the hydroxyapatite in the infected bone tissue and prevents bone implant related infection.

### CROSS REFERENCE TO RELATED PATENT APPLICATIONS

This patent application claims priority under 35 U.S.C. 119(e) to U.S. provisional patent application 62/031,989 entitled “Preparation and Characterization of Bone-Targeted Vancomycin-Loaded Liposomes for Osteomyelitis Treatment”, filed on Aug. 1, 2014. U.S. Provisional Patent Application Ser. No. 62/031,989 is incorporated herein by reference in its entirety.

### TECHNICAL FIELD

Embodiments are generally related to the targeting of vancomycin to bone tissue for the treatment of illness.

### BACKGROUND

Osteomyelitis is a bone infection that reaches the bone by traveling through the bloodstream or spreading from nearby tissue. Staphylococcus aureus is the most common cause of acute and chronic hematogenous osteomyelitis in adults and children. Increasingly, methicillin-resistant S. aureus (“MRSA”) is isolated from patients with osteomyelitis. The increasing prevalence of MRSA is making treatment more difficult. Surgical debridement is often necessary to remove dead bone tissue, followed by a long-term, high dose treatment with intravenous vancomycin.

Vancomycin is the drug of choice in the treatment of osteomyelitis caused mainly by MRSA. High doses of intravenous vancomycin as well as a prolonged duration of treatment are required to achieve therapeutic concentrations in bone tissue. This is primarily because of the poor blood supply to bone tissues. Serious side effects are normally associated with osteomyelitis therapy.

Therefore, a need exists for an improved Vancomycin delivery method, system, and compound to treat bone and joint infections.

### BRIEF SUMMARY

The following summary is provided to facilitate an understanding of some of the innovative features unique to the embodiments disclosed and is not intended to be a full description. A full appreciation of the various aspects of the embodiments can be gained by taking the entire specification, claims, drawings, and abstract as a whole.

It is, therefore, one aspect of the disclosed embodiments to provide medicinal treatment.

It is another aspect of the disclosed embodiments to provide methods and systems for treating bone and joint infections.

It is another aspect of the disclosed embodiments to provide a method and system for treating bone and joint infections using bone targeted liposomes.

It is another aspect of the disclosed embodiments to provide improved methods and systems for preparation and characterization of bone-targeted Vancomycin-loaded liposomes for treatment of osteomyelitis.

The aforementioned aspects and other objectives and advantages can now be achieved as described herein. In one embodiment, a treatment method comprises modifying at least one surface of at least one liposome to produce at least one surface-modified-liposome; targeting of infected bone tissue with the at least one surface-modified liposome; and preventing bone related infections by directly targeting the infected bone tissue with the at least one surface-modified liposome. Modifying the at least one surface of the at least one surface-modified liposome further comprises modifying the at least one surface of the at least one liposome with an alendronate targeting moiety.

In another embodiment, a liposome modification method comprises modifying a surface of at least one liposome to produce surface-modified liposomes; loading the surface-modified liposomes with vancomycin; maximizing an encapsulation efficiency of the vancomycin; and binding the surface-modified liposomes to hydroxyapatite crystals in bone tissue to thereby treat osteomyelitis-infected bone.

A liposome modification system comprises antibiotic-loaded, surface-modified liposomes that directly target bone tissue; a bisphosphonate as a targeting moiety, wherein the surface-modified liposomes bind preferably to hydroxyapatite crystals in bone tissue to treat osteomyelitis-infected bone. A dehydration and rehydration is used to maximize encapsulation efficiency of the antibiotic.

### BRIEF DESCRIPTION OF THE DRAWINGS

The accompanying figures, in which like reference numerals refer to identical or functionally-similar elements throughout the separate views and which are incorporated in and form a part of the specification, further illustrate the embodiments and, together with the detailed description, serve to explain the embodiments disclosed herein.

FIG. 1 illustrates an antibiotic delivery system in accordance with a preferred embodiment;

FIG. 2 illustrates a flow chart depicting operational steps of a method for the treatment of osteomyelitis in accordance with a preferred embodiment; and

FIG. 3 illustrates a flow chart depicting operational steps associated of a method for modifying a liposome in accordance with an alternative embodiment.

### DETAILED DESCRIPTION

The particular values and configurations discussed in these non-limiting examples can be varied and are cited merely to illustrate at least one embodiment and are not intended to limit the scope thereof. The embodiments will now be described more fully hereinafter with reference to the accompanying drawings, in which illustrative embodiments of the invention are shown. The embodiments disclosed herein can be embodied in many different forms and should not be construed as limited to the embodiments set forth herein; rather, these embodiments are provided so that this disclosure will be thorough and complete, and will fully convey the scope of the invention to those skilled in the art. Like numbers refer to like elements throughout. As used herein, the term “and/or” includes any and all combinations of one or more of the associated listed items.

The terminology used herein is for the purpose of describing particular embodiments only and is not intended to be limiting of the invention. As used herein, the singular forms “a”, “an”, and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The disclosed embodiments target vancomycin directly to bone tissue in order to treat or prevent bone infections, such as osteomyelitis, or bone implant related infections. In some embodiments, antibiotics such as daptomycin or other water-soluble antibiotics can be used for encapsulation. The antibiotic is encapsulated in a surface-modified liposome comprising an alendronate targeting moiety.

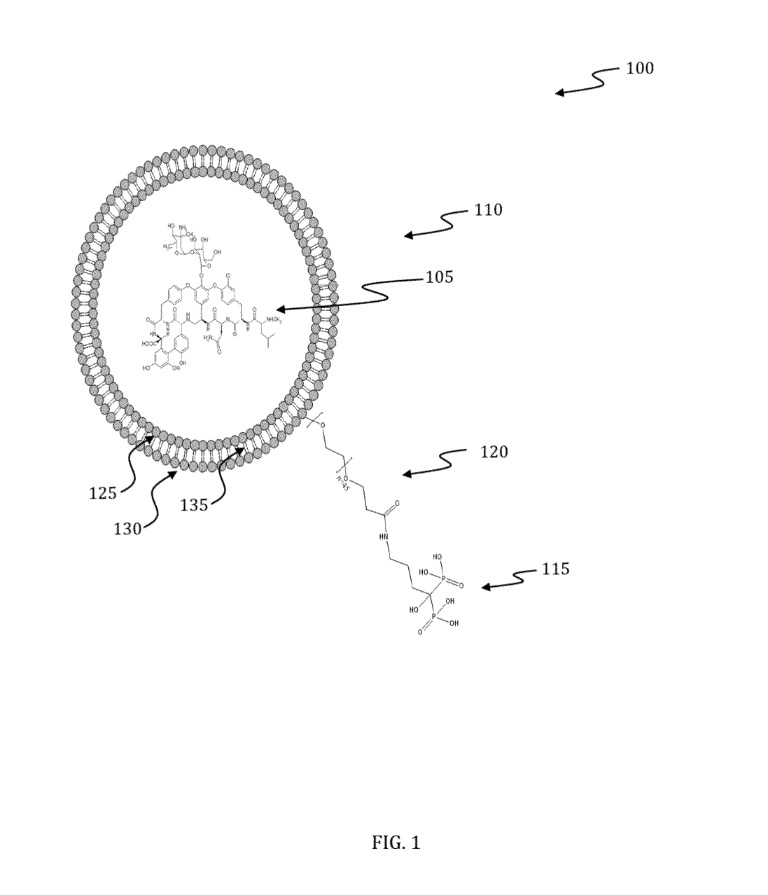

FIG. 1 illustrates an antibiotic delivery system 100 in accordance with a preferred embodiment. As shown in the FIG. 1, an antibiotic, in this case Vancomycin 105, can be encapsulated in a surface modified liposome 110 comprising an alendronate 115 targeting moiety 120. Liposomes, such as the liposome 110 depicted in FIG. 1, offer a safe and biocompatible mechanism to deliver a variety of therapeutic agents or antibiotics to a patient. Liposome 110 is a circular (or spherical) vesicle that includes a plurality of hydrophilic heads 130, a lipid bilayer 135, and a plurality of hydrophobic tails 125.

Liposomes generally have an aqueous solution core encapsulated by a lipid bilayer. The bilayer provides a shell through which hydrophilic solutes cannot pass. Thus, liposome 110 can be loaded with Vancomycin 105, or other such antibiotics so that they may be delivered to a desired location. The lipid bilayer 135 can attach to other cellular structures including, but not limited to, cell structures associated with bones. The liposome 110 can thus deliver the desired antibiotic to a target location. The liposome 110 disclosed herein may be considered a nano-carrier system. The liposome 110 provides a vehicle with which an antibiotic, such as Vancomycin 105, may be precisely delivered to targeted bone tissue.

Alendronate 115 is a bisphosphonate and an antiresorption drug that is currently used to treat osteoporosis. Bisphosphonates are generally understood to help decrease or prevent the loss of bone mass. In the embodiments disclosed herein, Alendronate 115 is preferred because of it has a strong binding affinity for the main component of bone tissue, specifically hydroxyapatite. This serves to enhance the targeted antibiotic delivery to bone tissue.

It should be understood that alendronate is one bisphosphonate that may be used for surface modification of liposome 110. However, it should be understood that surface modification of liposome 110 may also be achieved using any nitrogen containing biphsophonates such as alendronate, pamidronate, risedronate.

Embodiments disclosed herein therefore can include vancomycin-loaded, surface-modified liposomes 110 that directly target bone tissue. A modified dehydration-rehydration method can be used to maximize the encapsulation efficiency of Vancomycin 105 in the liposome 110.

Sodium alendronate can also be used as a targeting moiety. In particular, sodium alendronate can be conjugated to DSPE-PEG-COOH according to known methods. The conjugated DSPE-PEG-alendronate can then be used to prepare bone targeting vancomycin-loaded liposomes.

In-vitro binding test with hydroxyapatite showed that the surface modified liposomes 110 disclosed herein bind preferably to hydroxyapatite crystals, which are a major component of bone. Ex-vivo binding of the liposomes 110 to the tibia bone of four week old male CD1 mice indicated that the surface modified liposomes 110 disclosed herein bonded better to bone tissues as compare to the unmodified liposome.

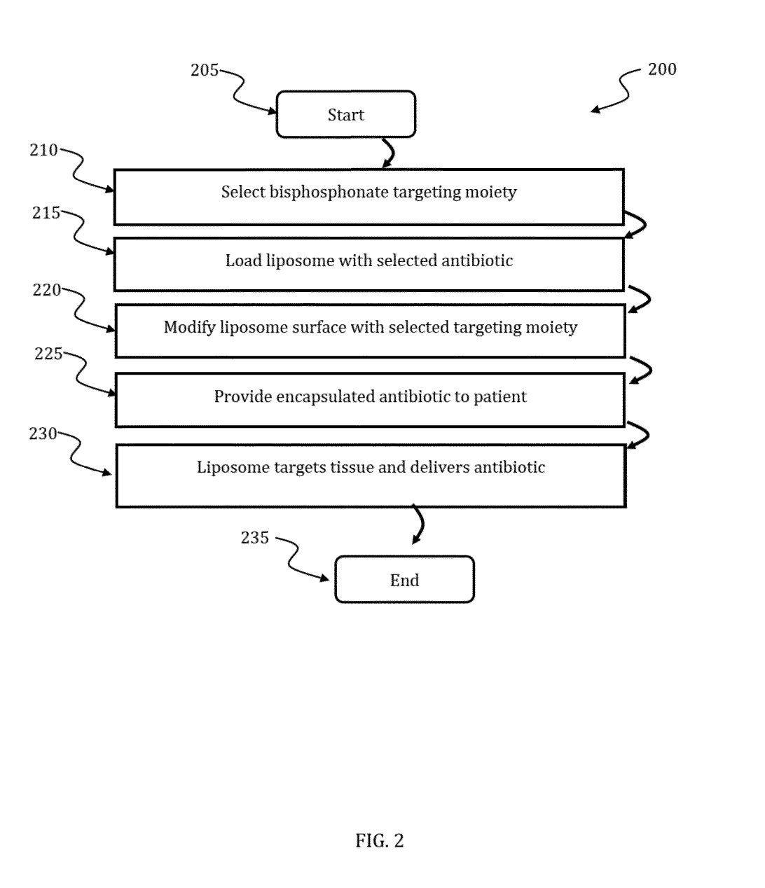

FIG. 2 illustrates a treatment method 200 in accordance a preferred embodiment. As shown in FIG. 2, the method 200 begins as indicated at step 205. As depicted at step 210, a targeting moiety can be selected for the desired bisphosphonate. This may be Alendronate or other such compound. As described thereafter at step 215, a selected antibiotic can be loaded into the liposome. The surface of the liposome can then be modified according to the selected targeting moiety as indicate at step 220 and then provided to the patient, as illustrated at step 225. The surface modified-liposome targets bone tissue thereby delivers the antibiotic to the targeted bone tissue, as shown at step 230. The method 200 then terminates as depicted at step 235.

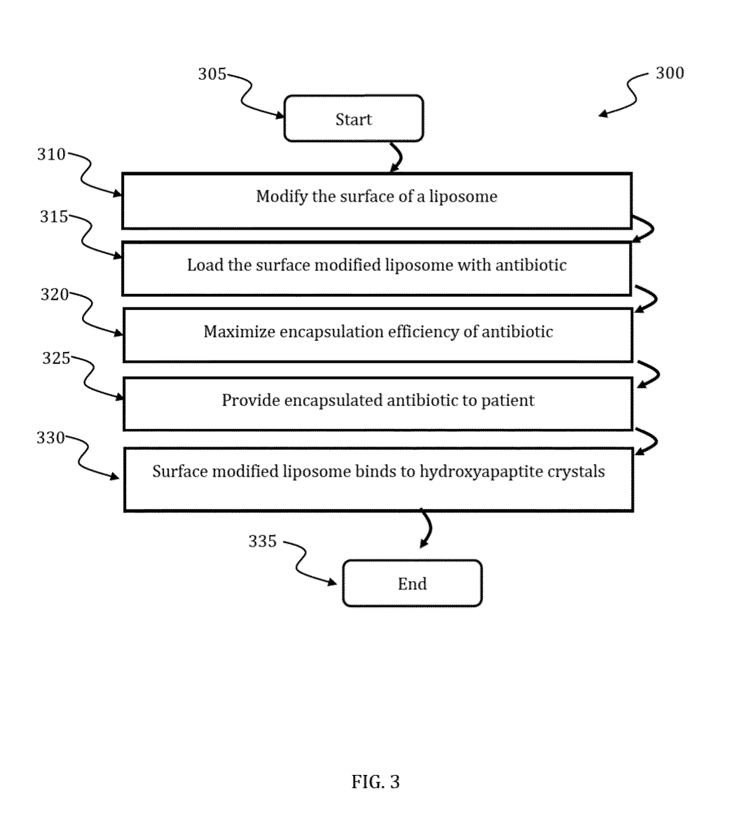

FIG. 3 illustrates a method 300 for modifying a liposome in accordance with an alternative embodiment. The method 300 begins as depicted at step 305. In the first step 310, the surface of a liposome can be modified with a bisphosponate. The bisphosphonate can be lendronate, pamidronate, risedronate; a nitrogen-containing bisphosphonate, or some combination thereof. Sodium alendronate may be used as a targeting moiety. The sodium alendronate can be conjugated to DSPE-PEG-COOH.

Next, as indicated at step 315 the surface-modified liposomes can be loaded with an antibiotic, which is preferably vancomycin, but may be other antibiotics such as daptomycin or other water-soluble antibiotics. The next step 320 involves maximizing the encapsulation efficiency of the antibiotic. This may be accomplished utilizing a dehydration and rehydration process.

Once the surface-modified liposome is loaded with an antibiotic, the surface-modified liposome may be administered to a patient, as described at step 325. The surface-modified liposome binds to the hydroxyapaptite crystals associated with the targeted bone tissue, delivering the encapsulated antibiotic to the desired bone tissue. The method 300 then terminates as indicated at step 335.

The disclosed embodiments combine surface modification of vancomycin-loaded liposomes with alendronate to utilize alendronate as a bone targeting agent, encapsulation of vancomycin within liposomes; and utilization of a surface modification technique to link alendronate to the liposomes in order to provide treatment of a desired tissue.

The embodiments disclosed herein result in decreased side effects as compared to traditional treatments, increase concentrations of the antibiotic in bone tissue, lower necessary doses, and more effectively treat osteomyelitis. The disclosed embodiments do not require local application of the antibiotic by direct implantation because the surface-modified liposomes directly targeted the bone from the blood stream.

Based on the foregoing, it can be appreciated that a number of embodiments, preferred and alternative, are disclosed herein. For example, in one embodiment, a treatment method comprises modifying at least one surface of at least one liposome to produce at least one surface-modified-liposome; targeting of infected bone tissue with the at least one surface-modified liposome; and preventing bone related infections by directly targeting the infected bone tissue with the at least one surface-modified liposome. Modifying the at least one surface of the at least one surface-modified liposome further comprises modifying the at least one surface of the at least one liposome with an alendronate targeting moiety.

In another embodiment, the treatment method further comprises targeting hydroxyapatite with the alendronate targeting moiety. The treatment method also comprises directly targeting hydroxyapatite with the at least one liposome. The at least one liposome comprises encapsulated vancomycin. In one embodiment, the infected bone tissue comprises osteomyelitis-infected bone tissue.

In another embodiment, modifying the at least one surface of the at least one surface-modified liposome further comprises modifying the at least one surface of the at least one liposome with a bisphosphonate targeting moiety. The treatment method further comprises targeting hydroxyapatite with the bisphosphonate targeting moiety.

In another related embodiment, the bisphosphonate targeting moiety comprises nitrogen containing bisphosphonate. The nitrogen containing bisphosphonate comprises at least one of alendronate; pamidronate; and risedronate. The at least one liposome comprises at least one encapsulated antibiotic configured to directly target the antibiotic to the hydroxyapatite.

In another embodiment, a liposome modification method comprises modifying a surface of at least one liposome to produce surface-modified liposomes; loading the surface-modified liposomes with vancomycin; maximizing an encapsulation efficiency of the vancomycin; and binding the surface-modified liposomes to hydroxyapatite crystals in bone tissue to thereby treat osteomyelitis-infected bone.

The method further comprises applying sodium alendronate as a targeting moiety, wherein alendronate is conjugated to DSPE-PEG-COOH, and utilizing the conjugated DSPE-PEG-alendronate to prepare bone targeting vancomycin-loaded liposomes.

In another embodiment, the method includes utilizing dehydration and rehydration to maximize encapsulation efficiency of the vancomycin.

In yet another embodiment, a liposome modification system comprises antibiotic-loaded, surface-modified liposomes that directly target bone tissue; a bisphosphonate as a targeting moiety, wherein the surface-modified liposomes hind preferably to hydroxyapatite crystals in bone tissue to treat osteomyelitis-infected bone. A dehydration and rehydration is used to maximize encapsulation efficiency of the antibiotic.

The system further comprises an alendronate wherein the alendronate is conjugated to DSPE-PEG-COOH, and wherein the conjugated DSPE-PEG-alendronate is used to prepare the surface-modified antibiotic-loaded liposomes. The antibiotic comprises at least one of vancomycin; daptomycin; and a water soluble antibiotic.

In another embodiment, the bisphosphonate comprises at least one of alendronate; pamidronate, risedronate, and a nitrogen-containing bisphosphonate.

It will be appreciated that variations of the above-disclosed and other features and functions, or alternatives thereof, may be desirably combined into many other different systems or applications. Also, it can be appreciated that various presently unforeseen or unanticipated alternatives, modifications, variations or improvements therein may be subsequently made by those skilled in the art which are also intended to be encompassed by the following claims.

What is claimed is:

1. A treatment method, comprising:
preparing at least one loaded liposome;
modifying at least one surface of said at least one liposome to produce at least one loaded surface-modified-liposome;
targeting of infected bone tissue with said at least one loaded surface-modified liposome; and
preventing bone related infections by directly targeting of said infected bone tissue with said at least one loaded surface-modified liposome.

2. The treatment method of claim 1 wherein modifying said at least one surface of said at least one loaded surface-modified liposome, further comprises:
modifying said at least one surface of said at least one loaded liposome with an alendronate targeting moiety.

3. The treatment method of claim 2 further comprising:
targeting hydroxyapatite with said alendronate targeting moiety.

4. The treatment method of claim 2 further comprising:
directly targeting hydroxyapatite with said at least one liposome.

5. The treatment method of claim 4 wherein said at least one liposome comprises encapsulated vancomycin.

6. The treatment method of claim 1 wherein said infected bone tissue comprises osteomyelitis-infected bone tissue.

7. The treatment method of claim 1 wherein modifying said at least one surface of said at least one surface-modified liposome further comprises:
modifying said at least one surface of said at least one liposome with a bisphosphonate targeting moiety.

8. The treatment method of claim 7 further comprising:
targeting hydroxyapatite with said bisphosphonate targeting moiety.

9. The treatment method of claim 7 wherein said bisphosphonate targeting moiety comprises nitrogen containing bisphosphonate.

10. The treatment method of claim 9 wherein said nitrogen containing bisphosphonate comprises at least one of:
alendronate:
pamidronate; and
risedronate.

11. The treatment method of claim 7 wherein said at least one liposome comprises at least one encapsulated antibiotic configured to directly target said antibiotic to said hydroxyapatite.

12. A liposome modification method comprising:
modifying a surface of at least one liposome to produce surface-modified liposomes;
loading said surface-modified liposomes with vancomycin;
maximizing an encapsulation efficiency of said vancomycin; and
binding said surface-modified liposomes to hydroxyapatite crystals in bone tissue to thereby treat osteomyelitis-infected bone.

13. The liposome modification method of claim 12 further comprising:
applying sodium alendronate as a targeting moiety, wherein alendronate is conjugated to DSPE-PEG-COOH.

14. The liposome modification method of claim 13 further comprising utilizing said conjugated DSPE-PEG-alendronate to prepare bone targeting vancomycin-loaded liposomes.

15. The liposome modification method of claim 12 further comprising:
utilizing dehydration and rehydration to maximize encapsulation efficiency of said vancomycin.

16. A liposome modification system, comprising:
antibiotic-loaded, surface-modified liposomes that directly target hone tissue; and
a bisphosphonate as a targeting moiety, wherein said surface-modified liposomes bind preferably to hydroxyapatite crystals in bone tissue to treat osteomyelitis-infected bone.

17. The liposome modification system of claim 16 wherein a dehydration and rehydration is used to maximize encapsulation efficiency of said antibiotic.

18. The liposome modification system of claim 16 further comprising:
an alendronate wherein said alendronate is conjugated to DSPE-PEG-COOH; and
wherein said conjugated DSPE-PEG-alendronate is used to prepare said surface-modified antibiotic-loaded liposomes.

19. The liposome modification system of claim 16 wherein said antibiotic comprises at least one of
vancomycin;
daptomycin; and
a water soluble antibiotic.

20. The liposome modification system of claim 16 wherein said bisphosphonate comprises at least one of:
alendronate;
pamidronate,
risedronate; and
a nitrogen-containing bisphosphonate.
